(19) United States
(12) Patent Application Publication
(10) Pub. No.: US 2004/0220084 A1
(43) Pub. Date: Nov. 4, 2004

(54) Methods for nucleic acid delivery

(76) Inventor: Jasbir Sandhu, Burlington (CA)

Correspondence Address: Michael A. Slavin, Esq., McHale & Slavin, P.A., Suite 402, 4440 PGA Boulevard, Palm Beach Gardens, FL, 33410, US

(21) Appl. No.: 10/429,660

(22) Filed: May 2, 2003

(51) Int. Cl. A61K 48/00

(52) U.S. Cl. 514/6; 514/44

(57) ABSTRACT

The invention provides methods useful for the multi-targeted delivery of nucleic acids to cells and tissues. The methods of the invention involve the administration of compositions containing at least one ligand such as human epidermal growth factor (EGF) or human vascular endothelial growth factor (VEGF), a nucleic acid and a human transferrin ligand to a host having cells to which nucleic acids can be delivered.

### CROSS REFERENCE TO RELATED APPLICATIONS

The instant application is related to application Ser. Nos. 10/______; 10/______; 10/______; 10/______; 10/______; 10/______; 10/_______; 10/______; 10/______; 10/______ and 10/______; all filed on even date herewith under Express Mail labels EV 140261687 US; EV 140261673 US; EV 140261660 US; EV 140261585 US; EV 140261571 US; EV 140261568 US; EV 140261554 US; EV 140261537 US; EV 140261523 US; EV 001630855 US and EV 001630864 US; the contents of which are each herein incorporated by reference.

### FIELD OF THE INVENTION

The instant invention relates generally to methods useful for the delivery of nucleic acids to cells and tissues; particularly to methods useful for multi-targeted delivery of nucleic acids to cells and tissues and most particularly to methods useful for multi-targeted delivery of nucleic acids involving the administration of compositions to hosts having cells to which nucleic acid can be delivered containing at least one ligand such as human epidermal growth factor (EGF) or human vascular endothelial growth factor (VEGF) and at least one nucleic acid each operatively linked to a human transferrin ligand.

### BACKGROUND OF THE INVENTION

The possibility of delivering foreign or heterologous genetic material into cells raises hope for the development of many new therapeutics. Prior artisans have produced compositions and methods useful for both in vitro and in vivo delivery of nucleic acids.

In order for efficient uptake of nucleic acid to occur, the nucleic acid must have direct contact with the cells to which it is to be delivered. Cells in vitro are easily physically accessible to direct contact through methods such as co-precipitation with calcium phosphate, electroporation, microinjection and particle bombardment (“gene gun”; firing of metallic microprojectiles coated with nucleic acid at the target cells). Cells in vivo are often not easily physically accessible to the direct contact necessary for efficient uptake of nucleic acids. Thus, although the above-referenced techniques are effective for in vitro use, they are impractical and potentially damaging for use in vivo.

Nucleic acids to be delivered to target cells in vivo are usually transported through the circulatory system where they are subject to damage and clearance from the body before ever reaching their target cells. Thus, carriers are employed for transport of nucleic acids in vivo in order to provide protection from damage and to prolong half-life. The efficiency of transport directly influences the resulting level of gene expression, meaning, for example, if transport is efficient, the uptake of nucleic acid is increased followed by an increase in gene expression. The ideal approach involves injection of nucleic acid complexed with a carrier into the circulation of a host followed by the “homing in” of the nucleic-acid carrier complex on the appropriate target cell to exclusion of all other cell types. However, to date, since most delivery methods lack selectivity, this “homing in” or targeting of nucleic acids to specific cells in vivo has proven to be an elusive goal for researchers. This difficulty of targeting nucleic acids to specific cells at the exclusion of all other cell types is a major barrier to progress in the development of effective in vivo delivery techniques. There are several broad categories of compositions and methods currently employed for in vivo delivery including; nucleic acid condensing agents, polymer systems, liposomes, viral vectors, and peptide-enhanced delivery methods.

For successful in vivo application it is important that nucleic acid delivery carriers be small enough to gain access to target cells by passage through cellular membranes. DNA can be condensed into small polyelectrolyte complexes by the addition of polycations, such as poly (L) lysine. These small complexes facilitate the transfer of DNA across the cell membrane for delivery to the nucleus. These polyelectrolyte complexes have shown limited usefulness due to rapid clearance from the circulation following intravenous injection. This rapid clearance results from destabilization of the polyelectrolyte complexes by serum proteins and degradation of the polyelectrolyte complexes by serum proteases.

Polymer systems have also been used for the delivery of nucleic acids, for example, by complexing nucleic acid with silica or by complexing nucleic acid with polymeric cations such as natural proteins and starches. However, these systems are lacking in selectivity and are subject to degradation and rapid clearance from the body.

Liposomes are phospholipid bilayer vesicles that can be used to encapsulate and transport nucleic acids. The nucleic acid is trapped in the aqueous compartment of the liposome and enclosed by the completely sealed lipid bilayer. The membranes of most cells have a net negative charge and thus the cationic nature of liposomes allows for attraction of the liposome to the cellular membrane resulting in fusion of the liposome with the cell membrane and subsequent release of the nucleic acid contained within. However, several disadvantages are associated with use of liposomes for the delivery of nucleic acids. Liposomes are not effective for nucleic acid delivery to all cell types. Often high doses of nucleic acid are required for successful delivery. Additionally, liposomes lack selectivity and can bind plasma proteins and the extracellular matrix thus never reaching their target cells.

Nucleic acids can also be integrated within a viral genome and delivered to target cells through viral infection. Adenovirus and retrovirus are commonly used for this purpose. The size of nucleic acid to be delivered by viral vector is limited to approximately 7-8 kb. Additionally, viral genes are also delivered with the nucleic acid which can lead to undesirable side effects including; immunogenicity, fixation of complement, poor-target selectivity and potential toxicity. It is also difficult to produce virus in the quantities necessary for use with delivery methods.

Delivery can also be enhanced by conjugation of the nucleic acid with specific peptides. Peptides containing aromatic amino acids (phenylalanine, tyrosine, tryptophan) are useful for this purpose. Additionally, viral peptides, such as fusogenic peptides derived from Staphylococcal Protein A enhance membrane fusion and thus can increase the efficiency of delivery. However, peptide-enhanced delivery is not effective for all types of cells.

Since many disadvantages remain associated with all of the compositions and methods currently employed for in vivo delivery discussed in the above paragraphs, there is clearly a need for improved compositions and methods for delivery of nucleic acids.

The delivery compositions and methods noted above exhibit a variety of disadvantages, however one disadvantage common to all is the lack of selectivity. Without selectivity delivery to specific cells is impossible. In an attempt to improve selectivity, researchers have attached molecules having the ability to target specific cell types to the surface of carriers, such as liposomes and polymeric cations. Examples of these types of molecules that can be attached to carriers are antibodies, cell adhesion peptides, hormones and cell-specific ligands. However, use of these molecules has not significantly improved selectivity of delivery.

Attachment of nucleic acids to multiple protein ligands that are each capable of specifically targeting cells is one approach to increasing selectivity of nucleic acid delivery methods, thereby increasing the efficiency of transport and thus increasing the resulting gene expression. A protein ligand is a soluble protein molecule that exhibits specific binding of high affinity for another molecule, for example, epidermal growth factor (EGF) is a ligand which specifically binds epidermal growth factor receptor (EGFR) on cellular surfaces with high affinity. Protein ligands are often internalized into the cell upon binding to their receptors, thus ligands can be used as vectors to carry nucleic acids specifically into target cells.

The present inventor has devised unique integrated moieties containing multiple ligands in linkage with at least one nucleic acid. These unique integrated moieties are capable of increasing the selectivity of nucleic acid delivery by specifically targeting multiple cell types using multiple ligands, thereby simultaneously increasing the efficiency of nucleic acid transport and the resulting gene expression. Use of these unique moieties for delivery of nucleic acids enables an increase in selectivity which represents a difference in kind as compared to the selectivity of delivery methods available in the prior art.

Although researchers have heretofore developed delivery methods utilizing compositions containing a single ligand linked to a carrier that targets an individual cell surface molecule, they have failed to produce delivery methods capable of increasing the selectivity of nucleic acid delivery by specifically targeting multiple cell types using multiple ligands, thereby simultaneously increasing the efficiency of nucleic acid transport and the resulting gene expression. What is lacking in the art is a delivery method capable of increasing the selectivity of nucleic acid delivery by specifically targeting multiple cell types using multiple ligands, thereby simultaneously increasing the efficiency of nucleic acid transport and the resulting gene expression.

### DESCRIPTION OF THE PRIOR ART

As is referred to above, there are several broad categories of compositions and methods currently employed for in vivo delivery of nucleic acids. Representative examples include:

U.S. Pat. No. 5,972,707 (Roy et al.) discloses a gene delivery system containing nanospheres made of enzymatically degradable polymeric cations complexed with nucleic acid. Targeting ligands can be attached directly or indirectly through a linking agent to the nanospheres disclosed by Roy et al. Roy et al. discloses a nanosphere with an attached transferrin ligand in Example 2 of U.S. Pat. No. 5,972,707. Roy et al. does not disclose or suggest attachment of multiple ligands capable of selectively targeting multiple cell types.

U.S. Pat. No. 6,051,429 (Hawley-Nelson et al.) discloses a composition useful for nucleic acid delivery wherein peptides are bound to the nucleic acid prior to addition of the transfection agent (cationic lipids or dendrimers). Hawley-Nelson et al. discloses four broad categories of peptides that can be used in their composition; fusagenic, membrane-permeabilizing, receptor-ligand and sub-cellular localization (nuclear or mitochondrial). These peptides increase the efficiency of transfection and can also be bound to the transfection agent according to the protocol taught by Hawley-Nelson et al. Hawley-Nelson et al. does not disclose or suggest attachment of multiple peptides capable of selectively targeting multiple cell types.

U.S. Pat. No. 6,312,727 (Schacht et al.) discloses a composition useful for delivery of nucleic acids containing synthetic polymer-based carriers. The carrier of Schacht et al. is made by self-assembly of the nucleic acid with cationic polymer material so as to condense the nucleic acid and form a polyelectrolyte complex and reacting the complex with a hydrophilic polymer material which binds to the complex thus stabilizing and protecting the complex. Schacht et al. discloses that transferrin can be added to the complex to increase the transfection efficiency. Schacht et al. does not disclose or suggest attachment of multiple molecules capable of selectively targeting multiple cell types.

U.S. Pat. No. 6,387,700 (Rice et al.) discloses a composition useful for delivery of nucleic acids containing low molecular weight nucleic acid condensing agents, such as aromatic amino acid (phenylalanine, tyrosine, tryptophan) containing peptides. Rice et al. disclose a general teaching wherein ligands selected to target specific cell surface receptors may be attached to the condensing agent in order to achieve site-specific targeting. However, Rice et al. does not disclose or suggest any specific types of ligands nor do they disclose or suggest the use of multiple ligands.

It is important to note that the delivery methods disclosed in the above references which use nucleic acid carriers with specific targeting molecules attached, involve a single ligand targeting a nucleic acid to a single receptor, this is in contrast to the delivery methods of the instant invention wherein multiple ligands target a nucleic acid to multiple receptors, thus enabling multiple cell types to produce the encoded protein. None of the above references disclose or suggest a delivery method capable of increasing the selectivity of nucleic acid delivery by specifically targeting multiple cell types using multiple ligands, thereby simultaneously increasing the efficiency of nucleic acid transport and the resulting gene expression.

### SUMMARY OF THE INVENTION

The instant invention provides delivery methods capable of increasing the selectivity of nucleic acid delivery by specifically targeting multiple cell types using multiple ligands, thereby simultaneously increasing the efficiency of nucleic acid transport and the resulting gene expression.

As used herein the term “nucleic acid” includes both DNA and RNA. Any nucleic acid is considered to be encompassed within the scope of the instant invention. The DNA sequences (SEQ ID NOS:9 and 11) encoding the human antibody against digoxin were used in the experimental examples herein described. However, it is noted that the use of the delivery methods of the instant invention to deliver DNA sequences encoding the human antibody against digoxin is an illustrative example only and is not intended to limit the methods to delivery of DNA sequences encoding the human antibody against digoxin. The compositions used in the delivery methods of the instant invention contain only human proteins and thus would not elicit an immune response when administered to a human host. The methods of the instant invention can be used to deliver any nucleic acid to a wide variety of cells and tissues without eliciting an immune response.

As used herein the term “ligand” refers to a molecule that exhibits specific binding of high affinity for another molecule and upon binding with that molecule is internalized into the cellular interior. Any such ligand is intended to be encompassed within the scope of the instant invention. Illustrative, albeit non-limiting examples of ligands known and commonly used in the art are antibodies, cell adhesion molecules, hormones and cell-specific ligands. Researchers can select ligands and nucleic acid sequences to design a composition for use when carrying out the methods of the instant invention according to need. Particularly preferred ligands for use are the cell-specific ligands, human epidermal growth factor (EGF), human vascular endothelial growth factor (VEGF) and human transferrin. The epidermal growth factor receptor (EGFR) is expressed by epithelial and epidermal cells and shows an increase in expression on the cellular surface of many types of tumor cells. The vascular endothelial growth factor receptor (VEGFR) is expressed by endothelial cells of non-resting blood vessels, which are particularly abundant in rapidly proliferating tumors. The transferrin receptor is ubiquitously expressed and shows an increase in expression on the cellular surface of actively proliferating cells, in particular in tumor tissues which often contain actively proliferating cells. Accordingly, EGF functions as a vector for delivery of nucleic acids to cells expressing the EGFR, VEGF functions as a vector for delivery of nucleic acids to cells expressing the VEGFR and transferrin functions as a vector for delivery of nucleic acids to cells expressing the transferrin receptor. Although each of these ligands (EGF, VEGF and transferrin) can be used to deliver nucleic acids to any cell expressing their respective complementary receptors (EGFR, VEGFR and the transferrin receptor), these ligands are particularly useful for cancer therapeutics, since these receptors are often highly expressed in tumor tissues.

Tumors are recognized as comprising a mixed population of cells including both neoplastic cells and normal endothelial cells. In order for a tumor to grow, new blood vessels are required to provide nutrients and to remove waste. Tumor cells secret growth factors to induce the formation of new blood vessels. These newly formed blood vessels comprise normal cells and are characterized by the expression of surface molecules that are not present on resting endothelium, for example vascular endothelial growth factor receptor (VEGFR). Additionally, the EGFR has been identified as a cell surface receptor that is overexpressed on many types of neoplastic cells and expression of the transferrin receptor is increased in actively proliferating cells, which may be both the neoplastic cells and endothelial cells of the tumor vasculature. Since certain particular embodiments of the instant invention comprise compositions containing ligands capable of targeting both neoplastic cells and the endothelial cells of the tumor vasculature, these methods can be used to deliver nucleic acids encoding therapeutic molecules to both cellular populations of the tumor mass.

Accordingly in certain embodiments, the compositions used in the methods of the instant invention contain a ligand such as human vascular endothelial growth factor (VEGF) or human epidermal growth factor (EGF) or contain both human VEGF and human EGF and at least one nucleic acid operatively linked to a human transferrin ligand, wherein said human VEGF binds to human VEGF receptors on endothelial cell surfaces of intratumoral blood vessels, said human EGF binds to human EGF receptors when present on cell surfaces of tumor cells and said human transferrin binds human transferrin receptors on endothelial cell surfaces of intratumoral blood vessels and cell surfaces of tumor cells. FIG. 1 shows a schematic diagram of several embodiments of the compositions used in the delivery methods described herein.

As used herein, the term vascular endothelial growth factor (VEGF) encompasses VEGF and isolated peptide fragments or biologically active portions thereof, analogues of VEGF and any biologically active portion thereof and any molecules and portions of molecules having the biological activity of VEGF.

As used herein, the term epidermal growth factor (EGF) encompasses EGF and isolated peptide fragments or biologically active portions thereof, analogues of EGF and any biologically active portion thereof and any molecules and portions of molecules having the biological activity of EGF.

As used herein, the term transferrin encompasses transferrin and isolated peptide fragments or biologically active portions thereof, analogues of transferrin and any biologically active portion thereof and any molecules and portions of molecules having the biological activity of transferrin.

As used herein, the term “bioactivity” refers to the ability of a ligand to bind to its complementary receptor thus enabling internalization of the ligand into the cellular interior.

As used herein, the term “biologically active portion” refers to the portion of a ligand that has the ability to bind to its complementary receptor thus enabling internalization of the ligand into the cellular interior.

When carrying out the methods of the instant invention the compositions used can be added to a pharmacologically effective amount of a carrier to provide pharmaceutical compositions for administration to an animal host, including administration to a human patient. Illustrative, albeit non-limiting examples of carriers known in the art and suitable for use with the instant invention are water, saline solutions and dextrose solutions. A particularly preferred carrier is saline, the use of which is illustrated in the examples herein.

Accordingly, it is an objective of the instant invention to provide a method for delivering nucleic acid to a cell of a host having cells to which nucleic acids can be delivered, said method comprising the steps of: (a) providing a compound comprising at least one ligand and at least one nucleic acid each operatively linked to transferrin, and (b) administering said compound to said cell whereby nucleic acid is delivered.

It is another objective of the instant invention to provide a method for delivering nucleic acid to a cell of a host having cells to which nucleic acids can be delivered, said method comprising the steps of: (a) providing a conjugate consisting essentially of at least one ligand and at least one nucleic acid each operatively linked to transferrin, and (b) administering said conjugate to said cell whereby nucleic acid is delivered.

It is another objective of the instant invention to provide a method for delivering nucleic acid to a cell of a host having cells to which nucleic acids can be delivered, said method comprising the steps of: (a) providing a compound comprising human vascular endothelial growth factor (VEGF) and at least one nucleic acid each operatively linked to human transferrin, and (b) administering said compound to said cell whereby nucleic acid is delivered.

It is yet another objective of the instant invention to provide a method for delivering nucleic acid to a cell of a host having cells to which nucleic acids can be delivered, said method comprising the steps of: (a) providing a conjugate consisting essentially of human vascular endothelial growth factor (VEGF) and at least one nucleic acid each operatively linked to human transferrin, and (b) administering said conjugate to said cell whereby nucleic acid is delivered.

It is another objective of the instant invention to provide a method for delivering nucleic acid to a cell of a host having cells to which nucleic acids can be delivered, said method comprising the steps of: (a) providing a compound comprising human epidermal growth factor (EGF) and at least one nucleic acid each operatively linked to human transferrin, and (b) administering said compound to said cell whereby nucleic acid is delivered.

It is another objective of the instant invention to provide a method for delivering nucleic acid to a cell of a host having cells to which nucleic acids can be delivered, said method comprising the steps of: (a) providing a conjugate consisting essentially of human epidermal growth factor (EGF) and at least one nucleic acid each operatively linked to human transferrin, and (b) administering said conjugate to said cell whereby nucleic acid is delivered.

It is yet another objective of the instant invention to provide a method for delivering nucleic acid to a cell of a host having cells to which nucleic acids can be delivered, said method comprising the steps of: (a) providing a compound comprising human vascular endothelial growth factor (VEGF), human epidermal growth factor (EGF) and at least one nucleic acid each operatively linked to human transferrin, and (b) administering said compound to said cell whereby nucleic acid is delivered.

It is another objective of the instant invention to provide a method for delivering nucleic acid to a cell of a host having cells to which nucleic acids can be delivered, said method comprising the steps of: (a) providing a conjugate consisting essentially of human vascular endothelial growth factor (VEGF), human epidermal growth factor (EGF) and at least one nucleic acid each operatively linked to human transferrin, and (b) administering said conjugate to said cell whereby nucleic acid is delivered.

It is still another objective of the instant invention to provide a method for delivering nucleic acid to a cell of a host having cells to which nucleic acids can be delivered, said method comprising the steps of: (a)providing a compound comprising at least one ligand, SEQ ID NO:9 and SEQ ID NO:11 each operatively linked to transferrin, and (b) administering said compound to said cell whereby nucleic acid is delivered.

It is another objective of the instant invention to provide a method for delivering nucleic acid to a cell of a host having cells to which nucleic acids can be delivered, said method comprising the steps of: (a) providing a conjugate consisting essentially of at least one ligand, SEQ ID NO:9 and SEQ ID NO:11 each operatively linked to transferrin, and (b) administering said conjugate to said cell whereby nucleic acid is delivered.

Other objectives and advantages of this invention will become apparent from the following description taken in conjunction with the accompanying drawings wherein are set forth, by way of illustration and example, certain embodiments of this invention. The drawings constitute a part of this specification and include exemplary embodiments of the present invention and illustrate various objects and features thereof.

### BRIEF DESCRIPTION OF THE FIGURES

FIG. 1 shows a diagrammatic presentation of several embodiments of the compositions used in the delivery methods described herein.

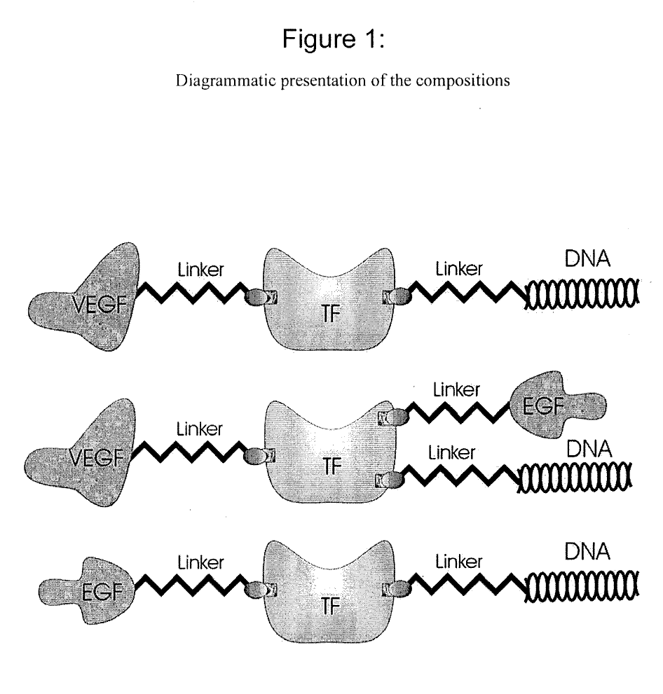

FIG. 2 shows a schematic presentation of the cloning steps necessary to produce the DNA vectors utilized in the experimental examples described herein.

FIG. 3 shows a graphical presentation of Breast Cancer Bone Metastatsis (BCBM) volumes in SCID mice.

FIGS. 4A-4B show immunohistochemistry of BCBM specific for EGFR (epidermal growth factor receptor). FIG. 4A shows a histologic section stained with antibody (TS40) specific for the human cell surface EGFR. FIG. 4B is a micrograph showing an isolated EGFR⁺ breast cancer cell in the bone marrow.

FIG. 5 is a micrograph showing blood vessels of human origin in the BCBM tumors in SCID mice.

FIG. 6 shows a graphical presentation of the serum level of anti-digoxin antibody achieved in the experimental SCID mice (non-tumor bearing mice).

### DEFINITIONS

The following list defines terms, phrases and abbreviations used throughout the instant specification. Although the terms, phrases and abbreviations are listed in the singular tense the definitions are intended to encompass all grammatical forms.

As used herein, the abbreviation “EGF” refers to epidermal growth factor.

As used herein, the abbreviation “EGFR” refers to epidermal growth factor receptor.

As used herein, the abbreviation “VEGF” refers to vascular endothelial growth factor.

As used herein, the abbreviation “VEGFR” refers to vascular endothelial growth factor receptor.

As used herein, the abbreviation “BCBM” refers to breast cancer bone metastatsis.

As used herein, the abbreviation “PEG” refers to polyethylene glygol.

As used herein, the abbreviation “TF” refers to transferrin.

As used herein, the abbreviation “SA” refers to streptavidin.

As used herein, the abbreviation “TF/SA” refers to a composition comprising transferrin linked to streptavidin.

As used herein, the abbreviation “MBS” refers to m-maleimidobenzoyl N-hydroxysuccinimide ester.

As used herein, the abbreviation “HPLC” refers to high performance liquid chromatography.

As used herein, the abbreviation “RP-HPLC” refers to reverse phase high performance liquid chromatography.

As used herein, the abbreviation “NHS” refers to N-hydroxysuccinimide.

As used herein, the abbreviation “TFA” refers to trifluoroacetic acid.

As used herein, the abbreviation “PBS” refers to phosphate buffered saline.

As used herein, the abbreviation “SCID” refers to a type of transgenic mouse that is severe combined immuno-deficient.

As used herein, the term “selective delivery” is defined as delivery which is targeted to a specific cell type for the purpose of avoiding uniform or even delivery to all cell types.

As used herein, the term “selective concentration” is defined as concentrating a substance, such as nucleic acid, to a specific area for the purpose of avoiding uniform or even concentration of a substance in all areas.

As used herein, the term “nucleic acid” is meant to encompass both DNA and RNA.

As used herein, the term “ligand” refers to a molecule that exhibits specific binding of high affinity for another molecule and upon binding with that molecule is internalized into the cellular interior. An illustrative, albeit non-limiting example of how the term “ligand” is used in the context of the instant specification is a protein ligand binding to a cell surface receptor, such as EGF binding to the EGFR.

As used herein, the term “receptor” refers to a molecule that exhibits specific binding of high affinity for its complementary ligand. An illustrative, albeit non-limiting example of how the term “receptor” is used in the context of the instant specification is a cell surface receptor binding to a ligand, such as the EGFR binding the EGF.

As used herein, the term “complementary receptor” refers to the receptor a ligand specifically binds with high affinity, for example, the EGFR is the complementary receptor for EGF.

As used herein, the term “target” refers to a specific molecule expressed on the cellular surface such as a receptor to which a specific moiety can be directed, for example the EGFR is a target for EGF. As used herein “target” can also refer to a cell or tissue, for example, a cell or tissue expressing the EGFR is a target for EGF.

As used herein, the term “targeting agent” refers to a specific molecule that binds to a complementary molecule expressed on the cellular surface such as a ligand, for example EGF is a targeting agent for the EGFR.

As used herein, the phrase “multi-targeted” refers to the ability of a protocol to target at least two disease elements, for example, the delivery methods of the instant invention can be used to target an entire tumor mass by using EGF to target the tumor cells (or by using VEGF to target the endothelial cells of the tumor vasculature) and by using transferrin to target both the tumor cells and the endothelial cells of the tumor vasculature.

As used herein, the phrase “disease elements” refers to the separate targets or elements that contribute to result in an entire disease state, for example, malignant cells and endothelial cells are each separate disease elements in cancer pathology.

As used herein, the term “VEGF” refers to a glycosylated polypeptide that serves as a mitogen to stimulate vascular development. VEGF imparts activity by binding to vascular endothelial cell plasma membrane-spanning tyrosine kinase receptors (VEGFR's) which then activates' signal transduction.

As used herein, the term “VEGFR” refers to a vascular endothelial cell plasma membrane-spanning tyrosine kinase receptor which binds VEGF thus exerting a mitogenic signal to stimulate vascularization of tissues.

As used herein, the term vascular endothelial growth factor (VEGF) encompasses VEGF and isolated peptide fragments or biologically active portions thereof, analogues of VEGF and any biologically active portion thereof and any molecules and portions of molecules having the biological activity of VEGF.

As used herein, the term “EGF” refers to a mitogenic polypeptide that exhibits growth stimulatory effects for epidermal and epithelial cells. EGF imparts activity by binding to epidermal and/or epithelial cell plasma membrane-spanning tyrosine kinase receptors (EGFR's) which then activates signal transduction.

As used herein, the term “EGFR” refers to a epidermal and/or epithelial cell plasma membrane-spanning tyrosine kinase receptor which binds EGF thus exerting a mitogenic signal.

As used herein, the term epidermal growth factor (EGF) encompasses EGF and isolated peptide fragments or biologically active portions thereof, analogues of EGF and any biologically active portion thereof and any molecules and portions of molecules having the biological activity of EGF.

As used herein, the term “transferrin” refers to a vertebrate glycoprotein that functions to bind and transport iron.

As used herein, the term “transferrin receptor” refers to a receptor expressed on the surface of cells which functions to bind iron saturated transferrin.

As used herein, the term transferrin encompasses transferrin and isolated peptide fragments or biologically active portions thereof, analogues of transferrin and any biologically active portion thereof and any molecules and portions of molecules having the biological activity of transferrin.

As used herein, the term “host” refers to any animal having cells to which nucleic acids can be delivered.

As used herein, the term “tumor tissue” refers to all of the cellular types which contribute to formation of a tumor mass, including tumor cells and endothelial cells, for example, the tumor tissue includes tumor cells and blood vessels.

As used herein, the term “tumor mass” refers to a foci of tumor tissue.

As used herein, the term “inhibition” refers to retarding the growth of a tumor mass.

As used herein, the term “bioactivity” refers to the ability of a ligand to bind to its complementary receptor thus enabling internalization of the ligand into the cellular interior.

As used herein, the term “biologically active portion” refers to the portion of a ligand that has the ability to bind to its complementary receptor thus enabling internalization of the ligand into the cellular interior.

As used herein, the phrases, “tumor vasculature”, “tumor endothelium” and “tumor vessels” all refer to the circulatory vessels which supply the tumor tissue with blood.

As used herein, the term “angiogenesis” refers to the process by which tissues become vascularized. Angiogenesis involves the proteolytic degradation of the basement membrane on which the endothelial cells reside followed by the chemotactic migration and mitosis of the endothelial cells to support a new capillary shoot.

As used herein, the term “linker” refers to the molecules which join the ligands of the composition used in the delivery methods of the instant invention together to form a single compound; for example, EGF-PEG attached to biotin links streptavidin attached to transferrin.

As used herein, the phrase “operatively linked” means that the linkage does not destroy the functions of each of the separate elements of the composition used in the delivery methods of the instant invention, for example, when linked together by a linker to form the single compound the ligands retain the ability to bind their complementary receptors.

As used herein, the term “carrier” refers to a pharmaceutically inert substance that facilitates delivery of an active agent to a host, for example, as is shown in the experiments described herein, saline functions as a carrier for delivery of the compositions used in the delivery methods of the instant invention to the mouse host.

As used herein, the phrase “pharmacologically effective amount of a carrier” refers to an amount of a carrier that is sufficient to effectively deliver an active agent to a host.

As used herein, the term “pharmaceutical composition” refers to the compositions used in the methods of the instant invention combined with a pharmacologically effective amount of a carrier.

The phrases “tumor endothelium”, “tumor vasculature” and “tumor vessels” are used interchangeably herein.

The terms “tumor cell”, “neoplastic cell” and “cancer cell” are used interchangeably herein.

As used herein, the term “compound” refers to a substance containing at least two distinct elements to which an unlimited number of other elements can be added.

As used herein, the term “conjugate” refers to a substance containing at least two distinct elements and a defined number of additional elements.

As used herein, the term “composition” is intended to encompass both a compound and a conjugate.

### DETAILED DESCRIPTION OF THE INVENTION

### Experimental Procedures

Sequences

The following nucleic acid sequences and corresponding amino acid sequences were used to generate the DNA and polypeptides used in the experiments described herein. Homo sapiens (human) VEGF165 (vascular endothelial growth factor isoform 165)nucleic acid sequence is disclosed as SEQ ID NO:1 and translates into VEGF165 protein disclosed as amino acid sequence SEQ ID NO:2. Homo sapiens (human) transferrin nucleic acid sequence is disclosed as SEQ ID NO:3 and translates into transferrin protein disclosed as amino acid sequence SEQ ID NO:4. Homo sapiens (human) EGF (epidermal growth factor) nucleic acid sequence is disclosed as SEQ ID NO:5 and translates into EGF protein disclosed as amino acid sequence SEQ ID NO:6. Homo sapiens (human) anti-digoxin antibody heavy chain nucleic acid sequence is disclosed as SEQ ID NO:9 and translates into anti-digoxin antibody heavy chain protein disclosed as SEQ ID NO:10. Homo sapiens (human) anti-digoxin antibody light chain nucleic acid sequence is disclosed as SEQ ID NO:11 and translates into anti-digoxin antibody light chain protein disclosed as SEQ ID NO:12.

Linkers

When assembling compositions from multiple elements, elements are either linked directly through chemical conjugation (for example through reaction with an amine or sulfhydryl group) or are linked indirectly through molecules termed linkers. When selecting a linker it is important to choose the appropriate length and flexibility of linker in order to reduce steric hindrance between the elements of the composition. For example, if an element of a composition is brought into close physical proximity of another element by linkage, the function of either or both elements can be affected. Each element of the composition must retain its bioactivity, for example in the instant invention, each ligand must retain its ability to bind to its complementary receptor after linkage with the other ligands of the composition. Illustrative, albeit non-limiting examples of linkers are glycols, alcohols and peptides. Particularly preferred linkers are PEG (polyethylene glycol) and the peptide linker shown as SEQ ID NO:8 (use of each of these linkers is illustrated in the examples described herein).

Crosslinking of VEGF (and EGF) to a Biotinylated-Polylinker

EGF and VEGF are crosslinked to a biotinylated polylinker by carrying out the following protocol. The polylinker used consists of 15 amino acid residues shown as SEQ ID NO:8. The cDNA sequence encoding this polylinker is shown as SEQ ID NO:7. The first glycine residue at the N-terminal was biotinylated. EDC (1-Ethyl-3-(3-Dimethylaminopropyl)carbodiimide Hydrochloride) and NHS (N-Hydroxysuccinimide) were equilibrated to room temperature. 0.4 mg of EDC and 0.6 mg of NHS were added to 1 mg/ml of the polylinker peptide solution (in activation buffer: 0.1 M MES (2-[N-morpholino]ethane sulfonic acid), 0.5 M NaCl, pH 6.0) to a final concentration of EDC and NHS of 2 mM and 5 mM respectively. The reaction mixture was then held for 15 minutes at room temperature. 1.4 ul of 2-mercaptoethanol was then added (to a final concentration of 20 mM). The reaction mixture was then run through P2 gel filtration mini-column and eluted by the activation buffer. Fractions containing the protein were then pooled together. Equal mole:mole ratios of either VEGF or EGF protein were added to the pooled fractions and reacted for 2 hours at room temperature. Hydroxylamine was added to a final concentration of 10 mM and the VEGF-linker or EGF-linker was purified by P2 gel filtration mini-column.

Synthesis of TF/SA Composition

8.84 mg of transferrin (TF) was thiolated by adding a 5-fold molar excess of 2-Iminothiolane hydrochloride (Traut's reagent) in pH 8.0, 0.16 M borate. Following 90 minutes at room temperature, the thiolated TF was desalted and concentrated by Centricon microconcentrators. Ellman's reagent (Pierce) was then used to demonstrate that a single thiol group was inserted on the surface of TF. 7 mg of streptavidin (SA) (in PBS) was activated by adding to a 20:1 molar ratio of m-maleimidobenzoyl N-hydroxysuccinimide ester (MBS)(stock at 1 mg/ml in dimethylformamide). After 20 minutes, the activated SA was desalted on a microconcentrator and immediately, the activated SA was added to a 10 molar excess of thiolated TF. They were mixed and then incubated at room temperature for 3 hours. Purification of the TF/SA composition was done by HPLC using TSK-G3000 column. The number of biotin binding sites per TF/SA composition was determined with ³H-biotin binding assay.

Conjugation of VEGF-Linker-Biotin (and EGF-Linker-Biotin) to TF-SA

VEGF-Linker-Biotin and EGF-Linker-Biotin are added to TF/SA by carrying out the following protocol. The composition of VEGF-Linker-biotin (or EGF-Linker-biotin) and TF/SA was prepared by mixing 5 nmol of VEGF-Linker-biotin (or 5 nmol of EGF-Linker-biotin) with 8 nmol of TF/SA (1:1.6 molar ratio). HPLC was then used to purify the VEGF-Linker-biotin-TF-SA composition (or EGF-Linker-biotin-TF-SA composition).

Conjugation of VEGF (and EGF) to PEG3400-Biotin

Alternatively to linkage with a peptide linker, VEGF and EGF can also be linked to transferrin using PEG by carrying out the following protocol. NHS-PEG3400-biotin was obtained from Shearwater Polymers (Huntsville, Ala.), where NHS=N-hydroxysuccinimide and PEG3400=poly(ethylene glycol) of 3400 Da molecular mass. NHS-PEG3400-biotin (20 nmol in 310 μl of 0.05 M NaHCO3) was added in a 1:1 molar ratio to either VEGF or EGF (16 nmol in 250 μl of 0.05 M NaHCO3) followed by incubation at room temperature for 60 minutes. The mixture was then applied to two Sepharose 12 HR 10/30 FPLC columns in series, followed by the elution in 0.01 M NaH2PO4/0.15 M NaCl/pH 7.5 at a flow rate of 0.7 mL/minute for 120 minutes. Fraction(s) that contained VEGF or EGF bound to PEG3400-biotin moiety were pooled together.

Conjugation of VEGF-PEG3400-Biotin (and EGF-PEG3400-Biotin) to VEGF-TF-SA (and/or EGF-TF-SA)

Following reaction of EGF and/or VEGF with NHS-PEG3400-biotin and transferrin with streptavidin, both compositions were purified by HPLC. The EGF (and/or VEGF)-NHS-PEG3400-biotin and TF/SA compositions were then mixed (1:1.6 molar ratio). HPLC was then used to purify the EGF (and/or VEGF)-NHS-PEG3400-biotin-TF-SA compositions.

Synthesis of Biotinylated Polylysine for Nucleic Acid Carrier and Attachment to TF

After attachment of the ligands(such as, EGF and VEGF) to the transferrin ligand, the nucleic acid was condensed and attached to the transferrin ligand according to the following protocol. Two polylysine (PLL) peptides were used for the DNA molecule to form a complex: Cys-Trp-Lys₁₉ (K₁₉, BioPeptide, San Diego, Calif.) and Lys₁₅₀ (K₁₅₀, average molecular weight of 20,000, Sigma, St Louis, Mo.). Biotin reagents for peptide modification were purchased from Pierce (Rockford, Ill.). Peptide K₁₉ was modified with a biotin group through the terminal cysteine residue by reaction of the sulfhydryl group with the iodoacetyl group of the biotinylation reagent, EZ-link-PEO-iodoacetyl-biotin. The peptide K₁₉ (10 mg) was dissolved in 90 μL of buffer (50 mM Tris, 5 mM EDTA, pH=8.3) that was previously bubbled with nitrogen gas. The EZ-link-PEO-iodoacetyl-biotin (48 mg) was also dissolved in 200 μL of buffer (0.1 M sodium phosphate, 5 mM EDTA, pH=6.0). The biotin solution was added dropwise to the peptide solution, mixed gently and incubated for 90 minutes. The starting peptide solution and the reaction mixture were analyzed by HPLC to determine if the reaction had gone to completion. The starting peptide solution and the reaction mixture were resolved by injecting 50 μg through a C18 RP-HPLC column eluted with water (0.1% trifluoroacetic acid) and an acetonitrile gradient (0.1% TFA, 0 to 95% over 50 minutes at 60° C.) while detecting the absorbance at 260 nm. For purification, sephadex (G15) was equilibrated in deionized water for 30 minutes prior to packing in a glass column (2 cm diameter×12 cm height). The reaction mixture was passed though the column using deionized water. Thirty fractions were collected, and the presence of the tryptophan side chain was examined by measuring the absorbance at 260 nm (Beckman Instruments Inc., Fullerton, Calif.). The fractions with the greatest absorbance at 260 nm were lyophilized. The purified biotinylated peptide (K₁₉-B) was stored as a powder at −20° C.

The peptide K₁₅₀ was biotinylated using succinimide ester (NHS)/amine chemistry. K₁₅₀ (10 mg) was dissolved in 1 mL of phosphate buffered saline (PBS, pH=7) and EZ-link-Sulfo-NHS-LC-biotin (2.8 mg) was added directly to the solution, mixed gently and incubated for 2 hours at 4° C. The reaction mixture was purified using dialysis cassettes immersed in deionized water. The dialyzed product was further purified using a monomeric avidin column to separate the biotinylated components from nonbiotinylated species. The biotinylated product was eluted with 10 mL of a 10 mM biotin solution and dialyzed to remove the unconjugated biotin. The purified biotinylated peptide (K₁₅₀-B) was then lyophilized and stored as a powder at −20° C.

The composition of polylysine-biotin and TF/SA was prepared by mixing polylysine-biotin with TF/SA (1:1.6 molar ratio). HPLC was then used to purify the polylysine-biotin-TF-SA composition. The reaction mixture was applied to a TSK-gel G3000 SWXL HPLC gel filtration column, followed by elution in 0.01 M Na₂HPO₄/0.15 M NaCl/pH 7.4/0.05% Tween-20 at a flow rate of 0.5 mL/min for 40 minutes, and 0.5 mL fractions were collected and lyophilized and stored as a powder at −20° C.

Digoxin is a cardiac glycoside found in the leaves of the foxglove plant group. Digoxin is used for long-term treatment of chronic heart weakness and defective heart valves. However, if administered in incorrect dosages, digoxin can be highly toxic. Anti-digoxin antibodies are often used to counter these toxic effects. (see Concise Encyclopedia Biochemistry and Molecular Biology, Third Edition, revised and expanded by Thomas A. Scott and E. Ian Mercer, Walter de Gruyter publisher, Berlin and New York, 1997, page 173, for a discussion of digoxin).

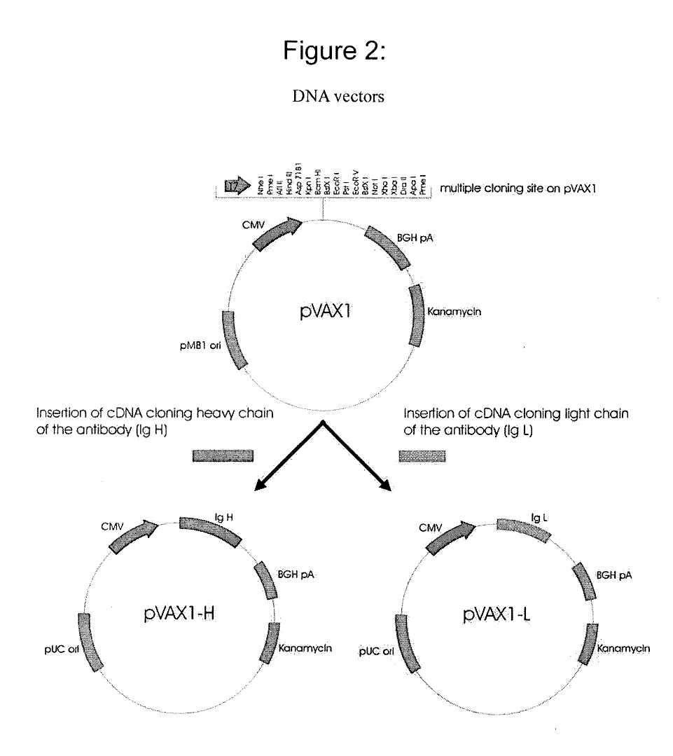

In order to test the efficacy of the delivery methods of the instant invention, nucleic acid encoding the human anti-digoxin antibody (IgG) was selected for delivery to SCID mice. Nucleic acid sequence, SEQ ID NO:9, encodes the heavy chain (including constant and variable regions) of the human anti-digoxin antibody and nucleic acid sequence, SEQ ID NO:11, encodes the light chain of the human anti-digoxin antibody (including constant and variable regions). FIG. 2 shows a schematic presentation of the cloning steps used to generate the DNA vectors containing SEQ ID NO:9 and SEQ ID NO:11 which were used in the experiments illustrated herein. SEQ ID NO:9 was inserted into the multiple cloning site of the pVAX1 plasmid to form the first vector (heavy chain) and SEQ ID NO:11 was inserted into the multiple cloning site of the pVAX1 plasmid to form the second vector (light chain). Symbols and abbreviations shown in FIG. 2 are defined as follows: IgH, refers to cDNA encoding the heavy chain of the immunoglobulin antibody against digoxin (SEQ ID NO:9); IgL, refers to cDNA encoding the light chain of the immunoglobulin antibody against digoxin (SEQ ID NO:11); T7, refers to the T7 promoter useful for achieving efficient transcription; CMV, refers to the human cytomegalovirus immediate-early promoter/enhancer useful for achieving efficient high-level expression of the sequences encoded in the multiple cloning site of the plasmids; BGH, refers to the bovine growth hormone polyadenylation (pA) signal useful for achieving efficient transcription termination and polyadenlylation of mRNA; kanamycin, refers to the gene encoding kanamycin resistance useful as a selection marker in E. coli to select cells expressing the cloned sequences and pUC ori, refers to the pUC-derived origin of replication useful for achieving high-copy number plasmid replication and growth in E. coli.

Following preparation of the vectors, 50 μg of each DNA vector (250 μg/ml) was mixed with polylysine-biotin-TF-SA.

Experimental Mice

Severe combined immuno-deficient C.B.-17 scid/scid (SCID) mice were bred and maintained according to the protocol of Sandhu et al. (Critical Reviews in Biotechnology 16(1):95-118 1996). Mice were used when 6-8 weeks old and were pre-treated with a dose of 3 Gy γ-radiation administered from a ¹³⁷CS source (Gamacell, Atomic Energy of Canada Ltd.Commercial Products). The irradiated SCID mice receive intraperitoneal injection of 20 μl ASGM1 sera diluted to 100 μl with saline, 4 hours pre-bone transplantation and every 7 days thereafter for the duration of the experiments.

The following groups of experiments were undertaken in order to determine the effectiveness of the EGF and VEGF ligands in cancer therapeutics.

Experimental Tumors

The delivery methods of the instant invention are effective when used to target either an EGFR⁺ tumor or an EGFR⁻ tumor since the transferrin moiety targets those tumor cells that are EGFR⁻. A bone metastatic focus of a primary EGFR⁺ breast tumor was used in the experimental examples herein described. However, it is noted that the use of the delivery methods of the instant invention in breast tumors is an illustrative example only and is not intended to limit the use of the methods to breast tumors. The methods of the instant invention can be used to deliver nucleic acids to any cells which are positive for the expression of at least one of the complementary receptors of the ligands contained in the compositions used in the delivery methods.

Implantation of Human Breast Cancer Bone Metastasis in SCID Mice

Breast cancer bone metastasis (BCBM) specimens (n=20, JJ1 to JJ20) were obtained from female patients (age range 40-68 years) undergoing total hip joint replacement due to BCBM mediated bone osteolysis. The majority (70%) of the BCBM used in these experiments were infiltrative ductal carcinoma and each specimen was assigned a number JJ1 to JJ20. Normal cancellous bone was obtained from healthy adult patients (age range 59-80 years) undergoing total hip joint replacement for the treatment of degenerative osteoarthritis. The BCBM was obtained from the proximal femur, morcellized using a rongeur and maintained under sterile conditions in RPMI (1640) medium (Gibco BRL, Burlington Ontario, Canada). Transplantation of the normal bone and BCBM into mice was performed within 2 hours of procurement, under a general anesthetic (intramuscular administration of Xylazine (4 μl/20 g mouse), and Ketamine (4 μl/20 g mouse) in 40 μl of 0.9% sodium chloride) under sterile conditions. Morcellized normal bone (Bone-SCID mice), and BCBM (BCBM-SCID mice), approximately 0.121 cm³ per mouse, was transplanted subcutaneously over the left flank in SCID mice (n=30).

Tumor Measurement

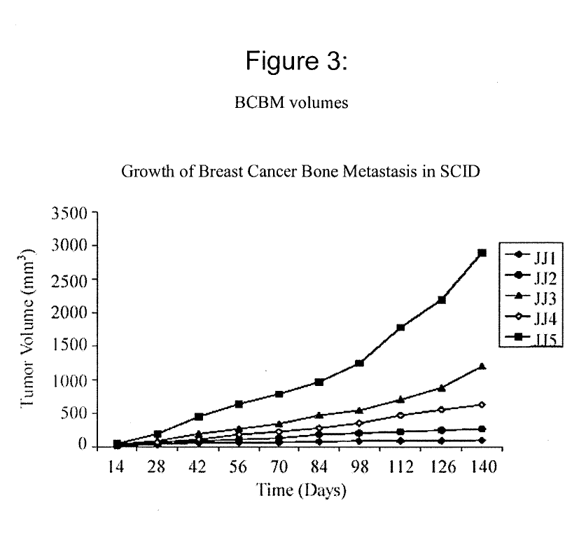

BCBM volumes were measured every 14 days for 20 weeks to assess tumor growth in SCID mice as described by Osborne et al. (Cancer Research 45:584-590 1985). The data shows that in contrast to the similar growth rate of breast cancer cell lines in immunodeficient mice the growth pattern of BCBM specimens varies in SCID mice (see FIG. 3). Results showed JJ5 gave the best growth of the tumor, thus it was chosen as the surgical specimen for use in subsequent in vitro cell studies and in vivo animal experiments.

Cell Culture Studies

Measurement of EGF-¹¹¹In-Labeled Transferrin Composition Binding to Breast Cancel Cells

Transferrin was radiolabled at the iron-binding sites in the following experiments. Methods of radiolabeling and ions useful for radiolabeling are commonly known in the art and one of ordinary skill in the art would be familiar with them.

Breast cancer cells express up to 100-fold higher levels of EGFR than do normal epithelial tissues. EGFR expression in breast cancer bone metastasis biopsies ranged from 1-1300 fmol/mg membrane protein (approximately 400-1,000,000 receptors/cell) and was associated with high relapse rate and poor long term survival. Normal epithelial cells express <10⁴ receptors/cell.

For the normal breast cell line HBL-100, 8000 EGFR/cell has been reported. The expression of EGFR in breast cancer cell lines has a reported range of 800 EGFR/cell for MCF-7 cells to 10⁶ EGFR/cell for MDA-MB-468 cells. The liver is the only normal tissue exhibiting moderate levels of EGFR (8×10⁴ to 3×10⁵ receptors/cell) likely reflecting its role in the elimination of EGF from the blood. Utilizing the Auger electron emitter ¹¹¹In was used in the initial experiments to illustrate the utility of the invention using EGF-111In-labeled transferrin compositions. The EGF-¹¹¹-In-labeled transferrin (0.25-80 ng) was incubated with 1.5×10⁶ cells/dish JJ5 Breast Cancer (prepared from BCBM JJ5) cells in 1 mL of 0.1% human serum albumin in 35 mm multiwell culture dishes at 37° C. for 30 minutes. The cells were transferred to a centrifuge tube and centrifuged. The cell pellet was separated from the supernatant and counted in a g-scintillation counter to determine bound (B) and free (F) radioactivity. Non-specific binding was determined by conducting the assay in 100 nM hEGF. The kinetics of binding was determined by incubating 1 ng of EGF-¹¹¹In-labeled transferrin composition with 3×10⁶ JJ5 Breast Cancer cells at 37° C. and determining the proportion of radioactivity bound to the cells at various times up to 24 hours. Internalized fraction was measured by determining the proportion of radioactivity which could not be displaced from the cell surface by 100 nM hEGF. Cell-associated binding (surface-binding and intracellular accumulation) was expressed as a percentage of medium radioactivity bound per mg of cell study protein.

The affinity constant for binding of EGF-¹¹¹In-labeled transferrin composition to JJ5 cells was 8×10⁸ L/mol and the number of binding sites was 2.7×10⁶. EGF-¹¹¹ In-labeled transferrin composition was rapidly bound by the breast cancer cells and retained for at least 24 hours. Over a 24 hour period at 37° C., <8% was lost from the cells in vitro.

The Growth Inhibition Assay of EGF-¹¹¹In-Labeled Transferrin Composition Against JJ5 Breast Cancer Cells

JJ5 breast cancer cells (prepared from BCBM JJ5) expressing approximately 10⁶ epidermal growth factor receptors/cell were incubated with EGF-¹¹¹In-labeled transferrin composition, unlabeled hEGF or ¹¹¹In-oxine, centrifuged to remove free ligand, then assayed and seeded (10⁶ cells/dish) into 35 mm culture dishes. Growth medium was added and the cells were cultured at 37° C./5% CO² for 4 days. The cells were then recovered by trypsinization and counted in a hemocytometer. Control dishes contained cells cultured in growth medium containing ¹¹¹In-DTPA or growth medium alone.

The growth inhibition assay of EGF-¹¹¹In-labeled transferrin composition (3.4 pCi/cell) achieved a 83% growth inhibition of the JJ5 cells compared to the medium control, whereas ¹¹¹In oxine (3.5 pCi/cell) which enters all the cells resulted in 91% growth inhibition.

Cytotoxicity Assay of EGF-¹¹¹In-Labeled Transferrin Composition Against JJ5 Breast Cancer Cells

JJ5 breast cancer cells were incubated with increasing amounts EGF-¹¹¹In-labeled transferrin composition or ¹¹¹In-oxine, centrifuged to remove free ligand, assayed and then seeded into 50 mm culture dishes. The number of cells seeded was varied from 3×10⁴ to 3×10⁶ cells to obtain approximately 400 viable colonies/dish taking into account the plating efficiency and the expected level of cytotoxicity. Control dishes contained JJ5 breast cancer cells which were incubated with normal saline. Growth medium was added and the cells were cultured at 37° C./5% CO² for 14 days. The growth medium was removed and the colonies were stained with methylene blue (1% in a 1:1 mixture of ethanol and water) then washed twice. The number of colonies per dish was counted using a manual colony counter (Manostat Corp). The plating efficiency was calculated by dividing the number of colonies observed by the number of cells seeded in each dish. The surviving fraction at increasing amounts of EGF-¹¹¹In-labeled transferrin composition or ¹¹¹In-oxine was calculated by dividing the plating efficiency for dishes containing treated cells with that observed for control dishes with normal saline.

Using a colony-forming assay, the radiotoxicity of internalization for JJ5 breast cancer cells was evaluated. EGF-¹¹¹In-labeled transferrin compositions (8 pCi/cell) resulted in a 99% reduction in cell survival for JJ5 cells. ¹¹¹In-oxine was also radiotoxic with greater than 99% cell killing at <6 pCi/cell.

There are various advantages of using the delivery methods of the instant invention in cancer therapy. As seen from the foregoing data, EGF-¹¹¹In-labeled transferrin compositions are rapidly internalized by cancer cells. The internalization process for EGF-¹¹¹In-labeled transferrin compositions involves an active transport mechanism utilizing the EGFR binding domain of the composition, rather than simple diffusion across the cell membrane. This active transport mechanism for the composition probably also includes nuclear translocation, as for the case of EGF, which allows for a maximal radiation dose of Auger electrons to be delivered to the cell's DNA. The compositions used in the delivery methods of the instant invention employ human polypeptides and are not immunogenic in humans. EGF-¹¹¹In-labeled transferrin compositions have been shown to retain ¹¹¹In over a 24 hour period at 37° C., with <8% of ¹¹¹In radioactivity was lost from cells in vitro. These characteristics are important for cell killing.

Immunohistochemistry Staining and Measurement of EGF Receptor on BCBM Cells

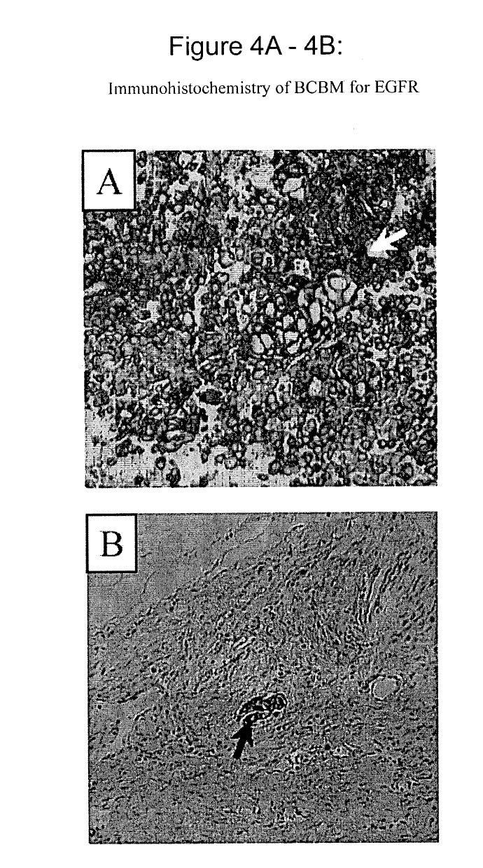

Immunohistochemistry of BCBM pre-implanted into mice showed all the specimens (n=20) had breast cancer cells negative for the estrogen and progesterone receptors (data not shown). Normal human bone histological sections were used as controls, no staining was observed in these specimens (data not shown). BCBM were retrieved from the mice at 20 weeks. Histologic sections were fixed and prepared. Immunohistochemical staining was done using mouse anti-EGF-receptor monoclonal antibody (TS40). In contrast to the implants and the controls, 16 of the 20 BCBM specimens had breast cancer cells positive for human EGFR (see FIGS. 4A-B). The white arrow in FIG. 4A points out a dense mass of EGFR⁺ cells. The arrow in FIG. 4B points out an isolated EGFR⁺ cell in the bone marrow. Mean (±SDEV) expression levels of EGF receptor was measured on breast cancer cells from tumor JJ5 by radioligand binding assay 24 and were in the range of 2.7 (±0.8)×10⁶ receptors/cell.

Immunohistochemistry Staining of BCBM Human Blood Vessels

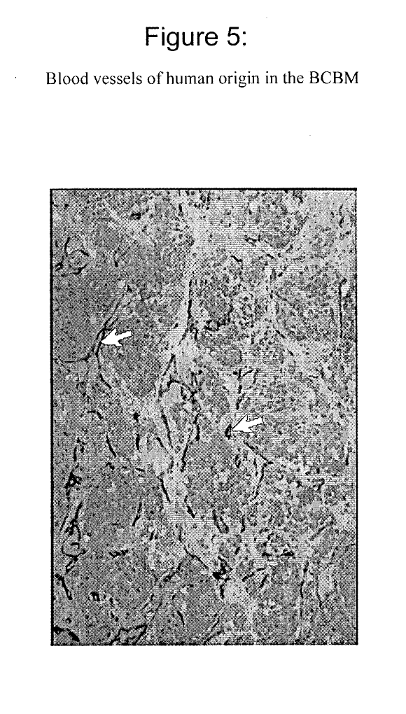

To evaluate the role of angiogenesis in the growth of human breast carcinoma, human BCBM surgical specimens were implanted in SCID mice. The breast tumors showed numerous blood vessels infiltrating the central mass of the tumors. In order to accurately assess the efficacy of treatment using the delivery methods of the instant invention against human tumors, the blood vessels which developed in the BCBM in the mice must be of human origin. Immunohistochemical staining was done on BCBM sections using mouse anti-human CD34 antibody. Anti-human CD34 reacts specifically with human blood vessels and thus will not react with murine blood vessels. As shown in FIG. 5, these results clearly demonstrate the presence of human blood vessel angiogenesis within the tumor xenografts retrieved from SCID mice at 20 weeks. In FIG. 5, the arrow points out the dark blood vessels of human origin (stained with anti-human CD34), thus these specimens can be used to accurately assess the efficacy of the VEGF portion of the composition used in the delivery methods of the instant invention.

Animal Studies

The following experiment was undertaken to demonstrate that the methods of the instant invention are capable of efficient delivery of nucleic acids to cells in vivo.

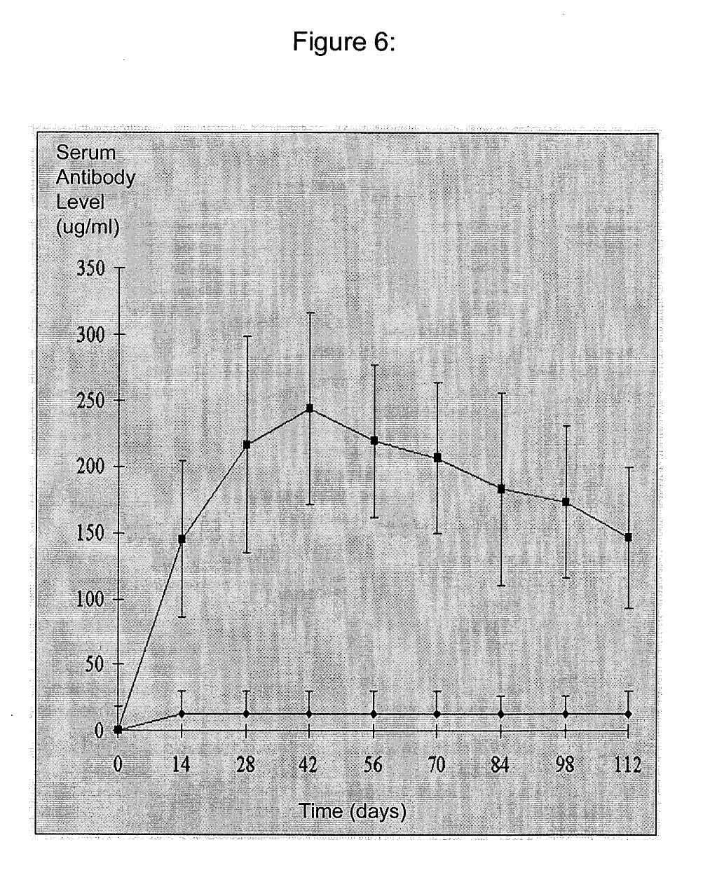

An EGF-PEG3400-biotin-TF-SA-DNA composition was prepared as described in the above experiments. The composition (250 μl) was injected intravenously into two groups of SCID mice; group one were normal SCID mice (non-tumor bearing) and group two were SCID mice implanted with JJ5 tumors (tumor-bearing). The control mice (also in two groups of tumor-bearing and non-tumor bearing) were administered the same amount of a similar composition, however the nucleic acid contained in this control composition did not encode for human anti-digoxin antibody. SCID mice serum was analyzed every two weeks using ELISA (Fishwild et al. Nature Biotechnology 14(7):845-851 1996) to determine the serum levels of human anti-digoxin antibody. Serum samples were exposed to plate-adsorbed digoxin-BSA, and biotinylated goat anto-mouse and anti-human heavy chain secondary antibodies were used to detect the hapeten-bound antibody. A strepavidin-alkaline phosphatase composition was used to quantitate anti-digoxin antibodies through hydrolysis of p-nitrophenylphosphate. FIG. 6 shows that the experimental group of mice produced the antibody for at least 112 days and in contrast very little antibody was detected in the serum of the control group of mice.

To examine if the anti-digoxin antibody seen in the serum was biologically active, mice survival studies using digoxin were done on all control and all experimental mice at 7 weeks post administration of the EGF-PEG3400-biotin-TF-SA-DNA composition. A general anesthetic [intramuscular administration of Xylazine (4 μl/20 g mouse), and Ketamine (4 μl/20 g mouse) in 40 μl of 0.9% sodium chloride] was administered to the mice under sterile conditions. The anesthetized mice were injected intravenously with 20 mg kg-1 of digoxin [purchased from Sigma USA] and animals were monitored continuously for 5 hours following digoxin administration. All the control mice (showing no serum anti-digoxin antibody) died from the digoxin overdose, in contrast the mice showing serum concentration of the anti-digoxin antibody survived the digoxin administration. These studies clearly demonstrate that the EGF-PEG3400-biotin-TF-SA-DNA composition of the instant invention is capable of efficient delivery of nucleic acids to cells in vivo and that the resulting protein produced in vivo is biologically active.

At the end of the digoxin-dosing experiments, DNA was isolated from the following tissues of the mice; lungs, liver, tumor, brain, kidney, blood, heart and muscle. Polymerase chain reaction was performed according to standard molecular biology protocols found in Molecular Cloning: A Laboratory Manual Authors: Joseph Sambrook and David W. Russell third edition 2001 on the isolated DNA for the presence of DNA coding for the anti-digoxin antibody. The antibody sequences were detected in the liver and the tumors in the tumor-bearing mice and only in the liver in the non-tumor bearing mice. This data suggests that the DNA is targeted to the liver by the VEGF, EGF and transferrin in the non-tumor bearing mice. In the tumor-bearing mice the VEGF, transferrin and EGF can target both the liver and tumor. Therefore this technology allows therapeutics coded by the DNA to be delivered directly to the tumors.

Antibodies of appropriate affinity and specificity have been shown capable of reversing advanced cardiac glycoside toxicity due to digoxin overdose. Without being bound by any particular theory, the reversal of digoxin effects by the digoxin antibody in the mice may be mediated by at least two mechanisms. First, the antibody may simply bind to cardiac glycoside and decrease the concentration of free digoxin in the serum, or second, the antibody may dislodge digoxin from its receptor.

In summary, the methods of the instant invention enable delivery of nucleic acids to at least two appropriate target cells in vivo to exclusion of all other cell types with the resulting production of a biologically active protein in the target cells. The delivery methods of the instant invention are amenable for use with any nucleic acid sequence of interest and permit the introduction of these sequences into a variety of cells and tissues, however, these delivery methods are particularly useful for introduction of such therapeutic molecules as antibodies and cytotoxic molecules without elicitation of an immune response. As is evidenced by the experimental examples described and shown herein, the instant invention provides delivery methods capable of increasing the selectivity of nucleic acid delivery by specifically targeting multiple cell types using multiple ligands, thereby simultaneously increasing the efficiency of nucleic acid transport and the resulting gene expression.

All patents and publications mentioned in this specification are indicative of the levels of those skilled in the art to which the instant invention pertains. All patents and publications are herein incorporated by reference to the same extent as if each individual patent and publication was specifically and individually indicated to be incorporated by reference.

It is to be understood that while a certain form of the invention is illustrated, it is not to be limited to the specific form or arrangement of parts herein described and shown. It will be apparent to those skilled in the art that various changes may be made without departing from the scope of the invention and the invention is not to be considered limited to what is shown and described in the specification.

One skilled in the art will readily appreciate that the present invention is well adapted to carry out the objects and obtain the ends and advantages mentioned, as well as those inherent therein. The oligonucleotides, peptides, polypeptides, biologically related compounds, methods, procedures and techniques described herein are presently representative of the preferred embodiments, are intended to be exemplary and are not intended as limitations on the scope. Changes therein and other uses will occur to those skilled in the art which are encompassed within the spirit of the invention and are defined by the scope of the appended claims. Although the invention has been described in connection with specific preferred embodiments, it should be understood that the invention as claimed should not be unduly limited to such specific embodiments. Indeed various modifications of the described modes for carrying out the invention which are obvious to those skilled in the art are intended to be within the scope of the following claims.

What is claimed is:

1. A method for delivering nucleic acid to a cell of a host having cells to which nucleic acids can be delivered, said method comprising the steps of:
(a) providing a compound comprising at least one ligand and at least one nucleic acid each operatively linked to transferrin, and
(b) administering said compound to said cell whereby nucleic acid is delivered.

2. A method in accordance with claim 1 wherein said at least one ligand is vascular endothelial growth factor (VEGF).

3. A method in accordance with claim 1 wherein said at least one ligand is epidermal growth factor (EGF).

4. A method for delivering nucleic acid to a cell of a host having cells to which nucleic acids can be delivered, said method comprising the steps of:
(a) providing a conjugate consisting essentially of at least one ligand and at least one nucleic acid each operatively linked to transferrin, and
(b) administering said conjugate to said cell whereby nucleic acid is delivered.

5. A method in accordance with claim 4 wherein said at least one ligand is vascular endothelial growth factor (VEGF).

6. A method in accordance with claim 4 wherein said at least one ligand is epidermal growth factor (EGF).

7. A method for delivering nucleic acid to a cell of a host having cells to which nucleic acids can be delivered, said method comprising the steps of:
(a) providing a compound comprising human vascular endothelial growth factor (VEGF) and at least one nucleic acid each operatively linked to human transferrin, and
(b) administering said compound to said cell whereby nucleic acid is delivered.

8. A method for delivering nucleic acid to a cell of a host having cells to which nucleic acids can be delivered, said method comprising the steps of:
(a) providing a conjugate consisting essentially of human vascular endothelial growth factor (VEGF) and at least one nucleic acid each operatively linked to human transferrin, and
(b) administering said conjugate to said cell whereby nucleic acid is delivered.

9. A method for delivering nucleic acid to a cell of a host having cells to which nucleic acids can be delivered, said method comprising the steps of:
(a) providing a compound comprising human epidermal growth factor (EGF) and at least one nucleic acid each operatively linked to human transferrin, and
(b) administering said compound to said cell whereby nucleic acid is delivered.

10. A method for delivering nucleic acid to a cell of a host having cells to which nucleic acids can be delivered, said method comprising the steps of:
(a) providing a conjugate consisting essentially of human epidermal growth factor (EGF) and at least one nucleic acid each operatively linked to human transferrin, and
(b) administering said conjugate to said cell whereby nucleic acid is delivered.

11. A method for delivering nucleic acid to a cell of a host having cells to which nucleic acids can be delivered, said method comprising the steps of:
(a) providing a compound comprising human vascular endothelial growth factor (VEGF), human epidermal growth factor (EGF) and at least one nucleic acid each operatively linked to human transferrin, and
(b) administering said compound to said cell whereby nucleic acid is delivered.

12. A method for delivering nucleic acid to a cell of a host having cells to which nucleic acids can be delivered, said method comprising the steps of:
(a) providing a conjugate consisting essentially of human vascular endothelial growth factor (VEGF), human epidermal growth factor (EGF) and at least one nucleic acid each operatively linked to human transferrin, and
(b) administering said conjugate to said cell whereby nucleic acid is delivered.

13. A method for delivering nucleic acid to a cell of a host having cells to which nucleic acids can be delivered, said method comprising the steps of:
(a) providing a compound comprising at least one ligand, SEQ ID NO:9 and SEQ ID NO:11 each operatively linked to transferrin, and
(b) administering said compound to said cell whereby nucleic acid is delivered.

14. A method in accordance with claim 13 wherein said at least one ligand is vascular endothelial growth factor (VEGF).

15. A method in accordance with claim 13 wherein said at least one ligand is epidermal growth factor (EGF).

16. A method for delivering nucleic acid to a cell of a host having cells to which nucleic acids can be delivered, said method comprising the steps of:
(a) providing a conjugate consisting essentially of at least one ligand, SEQ ID NO:9 and SEQ ID NO:11 each: operatively linked to transferrin, and
(b) administering said conjugate to said cell whereby nucleic acid is delivered.

17. A method in accordance with claim 16 wherein said at least one ligand is vascular endothelial growth factor (VEGF).

18. A method in accordance with claim 16 wherein said at least one ligand is epidermal growth factor (EGF).


SEQUENCE LISTING (12 sequences)

SEQ ID NO: 1 - DNA, 495 bp, Homo sapiens
gcacccatggcagaaggaggagggcagaatcatcacgaagtggtgaagttcatggatgtctatcagcgcagctactgccatccaatcgagaccctggtggacatcttccaggagtaccctgatgagatcgagtacatcttcaagccatcctgtgtgcccctgatgcgatgcgggggctgctgcaatgacgagggcctggagtgtgtgcccactgaggagtccaacatcaccatgcagattatgcggatcaaacctcaccaaggccagcacataggagagatgagcttcctacagcacaacaaatgtgaatgcagaccaaagaaagatagagcaagacaagaaaatccctgtgggccttgctcagagcggagaaagcatttgtttgtacaagatccgcagacgtgtaaatgttcctgcaaaaacacagactcgcgttgcaaggcgaggcagcttgagttaaacgaacgtacttgcagatgtgacaagccgaggcgg

SEQ ID NO: 2 - protein, 165 aa, Homo sapiens
APMAEGGGQNHHEVVKFMDVYQRSYCHPIETLVDIFQEYPDEIEYIFKPSCVPLMRCGGCCNDEGLECVPTEESNITMQIMRIKPHQGQHIGEMSFLQHNKCECRPKKDRARQENPCGPCSERRKHLFVQDPQTCKCSCKNTDSRCKARQLELNERTCRCDKPRR

SEQ ID NO: 3 - DNA, 2037 bp, Homo sapiens
gtccctgataaaactgtgagatggtgtgcagtgtcggagcatgaggccactaagtgccagagtttccgcgaccatatgaaaagcgtcattccatccgatggtcccagtgttgcttgtgtgaagaaagcctcctaccttgattgcatcagggccattgcggcaaacgaagcggatgctgtgacactggatgcaggtttggtgtatgatgcttacttggctcccaataacctgaagcctgtggtggcagagttctatgggtcaaaagaggatccacagactttctattatgctgttgctgtggtgaagaaggatagtggcttccagatgaaccagcttcgaggcaagaagtcctgccacacgggtctaggcaggtccgctgggtggaacatccccataggcttactttactgtgacttacctgagccacgtaaacctcttgagaaagcagtggccaatttcttctcgggcagctgtgccccttgtgcggatgggacggacttcccccagctgtgtcaactgtgtccagggtgtggctgctccacccttaaccaatacttcggctactcgggagccttcaagtgtctgaaggatggtgctggggatgtggcctttgtcaagcactcgactatatttgagaacttggcaaacaaggctgacagggaccagtatgagctgctttgcctagacaacacccggaagccggtagatgaatacaaggactgccacttggcccaggtcccttctcataccgtcgtggcccgaagtatgggcggcaaggaggacttgatctgggagcttctcaaccaggcccaggaacattttggcaaagacaaatcaaaagaattccaactattcagctctcctcatgggaaggacctgctgtttaaggactctgcccacgggtttttaaaagtccccccaaggatggatgccaagatgtacctgggctatgagtatgtcactgccatccggaatctacgggaaggcacatgcccagaagccccaacagatgaatgcaagcctgtgaagtggtgtgcgctgagccaccacgagaggctcaagtgtgatgagtggagtgttaacagtgtagggaaaatagagtgtgtatcagcagagaccaccgaagactgcatcgccaagatcatgaatggagaagctgatgccatgagcttggatggagggtttgtctacatagcgggcaagtgtggtctggtgcctgtcttggcagaaaactacaataagagcgataattgtgaggatacaccagaggcagggtattttgctgtagcagtggtgaagaaatcagcttctgacctcacctgggacaatctgaaaggcaagaagtcctgccatacggcagttggcagaaccgctggctggaacatccccatgggcctgctctacaataagatcaaccactgcagatttgatgaatttttcagtgaaggttgtgcccctgggtctaagaaagactccagtctctgtaagctgtgtatgggctcaggcctaaacctgtgtgaacccaacaacaaagagggatactacggctacacaggcgctttcaggtgtctggttgagaagggagatgtggcctttgtgaaacaccagactgtcccacagaacactgggggaaaaaaccctgatccatgggctaagaatctgaatgaaaaagactatgagttgctgtgccttgatggtaccaggaaacctgtggaggagtatgcgaactgccacctggccagagccccgaatcacgctgtggtcacacggaaagataaggaagcttgcgtccacaagatattacgtcaacagcagcacctatttggaagcaacgtaactgactgctcgggcaacttttgtttgttccggtcggaaaccaaggaccttctgttcagagatgacacagtatgtttggccaaacttcatgacagaaacacatatgaaaaatacttaggagaagaatatgtcaaggctgttggtaacctgagaaaatgctccacctcatcactcctggaagcctgcactttccgtagacct

SEQ ID NO: 4 - protein, 679 aa, Homo sapiens
VPDKTVRWCAVSEHEATKCQSFRDHMKSVIPSDGPSVACVKKASYLDCIRAIAANEADAVTLDAGLVYDAYLAPNNLKPVVAEFYGSKEDPQTFYYAVAVVKKDSGFQMNQLRGKKSCHTGLGRSAGWNIPIGLLYCDLPEPRKPLEKAVANFFSGSCAPCADGTDFPQLCQLCPGCGCSTLNQYFGYSGAFKCLKDGAGDVAFVKHSTIFENLANKADRDQYELLCLDNTRKPVDEYKDCHLAQVPSHTVVARSMGGKEDLIWELLNQAQEHFGKDKSKEFQLFSSPHGKDLLFKDSAHGFLKVPPRMDAKMYLGYEYVTAIRNLREGTCPEAPTDECKPVKWCALSHHERLKCDEWSVNSVGKIECVSAETTEDCIAKIMNGEADAMSLDGGFVYIAGKCGLVPVLAENYNKSDNCEDTPEAGYFAVAVVKKSASDLTWDNLKGKKSCHTAVGRTAGWNIPMGLLYNKINHCRFDEFFSEGCAPGSKKDSSLCKLCMGSGLNLCEPNNKEGYYGYTGAFRCLVEKGDVAFVKHQTVPQNTGGKNPDPWAKNLNEKDYELLCLDGTRKPVEEYANCHLARAPNHAVVTRKDKEACVHKILRQQQHLFGSNVTDCSGNFCLFRSETKDLLFRDDTVCLAKLHDRNTYEKYLGEEYVKAVGNLRKCSTSSLLEACTFRRP

SEQ ID NO: 5 - DNA, 159 bp, Homo sapiens
aactctgattccgaatgcccgctgtctcatgacggttactgcctgcatgatggcgtatgcatgtacatcgaagctctggacaaatacgcatgcaactgtgttgtaggttacatcggcgaacgttgccagtatcgcgacctgaaatggtgggaactgcgt

SEQ ID NO: 6 - protein, 53 aa, Homo sapiens
NSDSECPLSHDGYCLHDGVCMYIEALDKYACNCVVGYIGERCQYRDLKWWELR

SEQ ID NO: 7 - DNA, 45 bp, Artificial
  sequence codes for a polylinker
ggtggcggtggctcgggcggtggtgggtcgggtggcggcggatct

SEQ ID NO: 8 - protein, 15 aa, Artificial
  sequence of a polylinker
GGGGSGGGGSGGGGS

SEQ ID NO: 9 - DNA, 1407 bp, Homo sapiens
atggctgtcctggtgctgttcctctgcctggttgcatttccaagctgtgtcctgtcccaggtgcacctgaaggagtcaggacctggcctggtggcgccctcacagagcctgtccatcacttgcactgtctctggattttcattaaccacctatggtgtacactggtttcgccagcctccaggaaagggtctggagtggctgggactaatatgggctggtggaaacacagattataattcggctctcatgtccagactgagcatcaacaaagacaactccaagagccaagttttcttaaaaatgaacagtctgcaagctgatgacacagccatgtactactgtgccagatttcgctttgcttcttactacgactatgctgtggactactggggtcaaggaacctcagtcaccgtctcctcatccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtgcacaccttcccggctgtcctacagtcctcaggactctactccctcagcagcgtggtgaccgtgccctccagcagcttgggcacccagacctacatctgcaacgtgaatcacaagcccagcaacaccaaggtggacaagaaagttgagcccaaatcttgtgacaaaactcacacatgcccaccgtgcccagcacctgaactcctggggggaccgtcagtcttcctcttccccccaaaacccaaggacaccctcatgatctcccggacccctgaggtcacatgcgtggtggtggacgtgagccacgaagaccctgaggtcaagttcaactggtacgtggacggcgtggaggtgcataatgccaagacaaagccgcgggaggagcagtacaacagcacgtaccgggtggtcagcgtcctcaccgtcctgcaccaggactggctgaatggcaaggagtacaagtgcaaggtctccaacaaagccctcccagcccccatcgagaaaaccatctccaaagccaaagggcagccccgagaaccacaggtgtacaccctgcccccatcccgggatgagctgaccaagaaccaggtcagcctgacctgcctggtcaaaggcttctatcccagcgacatcgccgtggagtgggagagcaatgggcagccggagaacaactacaagaccacgcctcccgtgctggactccgacggctccttcttcctctacagcaagctcaccgtggacaagagcaggtggcagcaggggaacgtcttctcatgctccgtgatgcatgaggctctgcacaaccactacacgcagaagagcctctccctgtctccgggtaaa

SEQ ID NO: 10 - protein, 469 aa, Homo sapiens
MAVLVLFLCLVAFPSCVLSQVHLKESGPGLVAPSQSLSITCTVSGFSLTTYGVHWFRQPPGKGLEWLGLIWAGGNTDYNSALMSRLSINKDNSKSQVFLKMNSLQADDTAMYYCARFRFASYYDYAVDYWGQGTSVTVSSSTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - DNA, 708 bp, Homo sapiens
atggagacagacacactcctgttatgggtactgctgctctgggttccaggttccactggtgacattgtgctgacacagtctcctgcttccttagctgtatctctggggcagagggccaccatctcatgcagggccagcaaaagtgtcagtacatctggctatagtcatatacactggtaccaacagaaaccaggacagccacccaaactcctcatctatcttgcatccatcctagaatctggggtccctgccaggttcagtggcagtgggtctgggacagacttcaccctcaacatccatcctgtggaggaggaggatgctgcaacctattactgtcaacacagtagggaatatccgctcacgttcggtgctgggaccgagctggagctgaaacagcccaaggctgccccctcggtcactctgttcccgccctcctctgaggagcttcaagccaacaaggccacactggtgtgtctcataagtgacttctacccgggagccgtgacagtggcctggaaggcagatagcagccccgtcaaggcgggagtggagaccaccacaccctccaaacaaagcaacaacaagtacgcggccagcagctatctgagcctgacgcctgagcagtggaagtcccacagaagctacagctgccaggtcacgcatgaagggagcaccgtggagaagacagtggcccctacagaatgttca

SEQ ID NO: 12 - protein, 236 aa, Homo sapiens
METDTLLLWVLLLWVPGSTGDIVLTQSPASLAVSLGQRATISCRASKSVSTSGYSHIHWYQQKPGQPPKLLIYLASILESGVPARFSGSGSGTDFTLNIHPVEEEDAATYYCQHSREYPLTFGAGTELELKQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS